(19) United States
(12) Patent Application Publication
(10) Pub. No.: US 2024/0032876 A1
(43) Pub. Date: Feb. 1, 2024

(54) SYSTEMS AND METHODS FOR DISPLAYING ANALYTE LEVELS

(71) Applicant: GLOWCOSE LLC, Charlotte, NC (US)

(72) Inventor: Kevin Terry, Charlotte, NC (US)

(21) Appl. No.: 18/360,037

(22) Filed: Jul. 27, 2023

Related U.S. Application Data
(60) Provisional application No. 63/393,147, filed on Jul. 28, 2022.

(51) Int. Cl. A61B 5/00; A61B 5/145

(52) U.S. Cl. CPC A61B 5/7445; A61B 5/14532

(57) ABSTRACT

A method is provided that may include determining an analyte concentration and determining one or more concentration ranges based on the analyte concentration. The one or more concentration ranges may include a low level concentration range, a target level concentration range, and a high level concentration range. The method may include communicating the one or more concentration ranges to an adjustable display and changing a hue displayed by the display based on the one or more concentration ranges.

### CROSS-REFERENCE TO RELATED APPLICATIONS

This application claims priority to U.S. Provisional Application No. 63/393,147, filed on Jul. 28, 2022, the entire disclosure of which is incorporated herein by reference.

### BACKGROUND

### Technical Field

Examples of the subject matter herein relate to methods and systems of processing and displaying measured analyte levels, such as blood glucose levels.

### Discussion of Art

Various conditions may require a user to monitor the level of a particular analyte in their body. For example, diabetes mellitus is a disorder in which the pancreas cannot create sufficient insulin (Type I or insulin dependent) and/or in which insulin is not effective (Type 2 or non-insulin dependent). Where blood sugar levels get too high or too low, there can be serious and even fatal health outcomes.

Conventionally, a diabetic person carries a self-monitoring blood glucose (SMBG) monitor, which typically requires uncomfortable finger pricking methods. Due to the lack of comfort and convenience, a diabetic may normally only measure his or her glucose level two to four times per day. Unfortunately, these time intervals may be spread so far apart that the diabetic may find out too late, sometimes incurring dangerous side effects, of a hyperglycemic or hypoglycemic condition. In fact, it may not be only unlikely that a diabetic will take a timely SMBG value, but additionally the diabetic may not know if their blood glucose value may be going up (higher) or down (lower) based on conventional methods.

Another device that some diabetics use to monitor their blood glucose is a continuous analyte sensor, e.g., a continuous glucose monitor (CGM). A CGM typically includes a sensor that may be placed invasively, minimally invasively or non-invasively. The sensor may measure the concentration of a given analyte within the body, e.g., glucose, and may generate a raw signal that may be generated by electronics associated with the sensor. The raw signal may be converted into an output value that may be displayed on a display. The output value that results from the conversion of the raw signal may typically be expressed in a form that provides the user with meaningful information, and in which form users become familiar with analyzing, such as blood glucose expressed in mg/dL.

CGM may send the output value to an app on a mobile device to allow the user to view their blood glucose level. However, it may be advantageous to the user to have a more accessible, visible, and discreet way of reading their blood glucose level that does not require pulling out their phone and opening an app. It may be important to have a visual representation of analyte levels that is easily accessible, visible, easy to understand, and adjustable.

It may be desirable to have a system and method that differs from those that are currently available.

### BRIEF DESCRIPTION

In accordance with one example or aspect, a method is provided that includes determining an analyte concentration. The method may further include determining concentration ranges based on the analyte concentration. The concentration ranges may include a low level concentration range, a target level concentration range, and a high level concentration range. The method may include communicating the concentration range to an adjustable display. The method may include changing a hue displayed by the adjustable display based on the concentration range.

In accordance with one example or aspect, a method is provided that includes determining an analyte concentration. The method may include communicating the analyte concentration to an adjustable display. The method may include modifying a hue displayed by the adjustable display based on the analyte concentration.

In accordance with one example or aspect, a system is provided that includes an adjustable light display and a controller. The adjustable light display may include one or more processors. The controller may be in communication with the adjustable light display. The controller may retrieve a measured analyte reading from a database and communicate the measured analyte reading to the one or more processors. The one or more processors may determine a hue of the adjustable light display based on the measured analyte reading. The controller may change the hue of the adjustable light display based on the measured analyte reading.

### BRIEF DESCRIPTION OF THE DRAWINGS

The subject matter may be understood from reading the following description of non-limiting examples, with reference to the attached drawings, wherein below:

FIG. 1 illustrates a system for displaying measured analyte levels, according to one example;

FIG. 2 illustrates a system for displaying measured analyte levels, according to one example;

FIG. 3 illustrates a side view of a system for displaying measured analyte levels, according to one example;

FIG. 4 illustrates a rear view of the system for displaying measured analyte levels illustrated in FIG. 3, according to one example;

FIG. 5 illustrates a color wheel, according to one example;

FIG. 6 illustrates an application dashboard, according to one example;

FIG. 7 illustrates a range setup of the application dashboard of FIG. 6, according to one example; and

FIG. 8 illustrates a flowchart of a method displaying analyte levels, according to one example.

### DETAILED DESCRIPTION

Embodiments of the subject matter described herein relate to methods and systems for processing and displaying measured analyte levels, such as blood glucose levels. A user may monitor their blood glucose readings using a continuous glucose monitor (CGM). The CGM may communicate a sensor reading, such as a blood glucose reading to a transmitter. The transmitter may then transmit the blood glucose levels detected by the CGM to a receiver or monitor unit. The transmitter may use wireless communication, such as radio frequency, Bluetooth, Wi-Fi, or the like. In some CGMs, the blood glucose reading may be stored in a cloud storage system and may be communicated from the cloud storage system to a mobile app for access by the user.

While reading blood glucose levels by opening a mobile app may be an improvement over past iterations of blood glucose monitoring, it may be advantageous to have blood glucose readings be more accessible, visible, and discreet for the user. For example, when monitoring blood glucose levels at night, opening a phone and navigating to the mobile app may be cumbersome and disruptive. Additionally, monitoring blood glucose levels in children may be difficult using a mobile app as the child may not have a mobile device or may not be comfortable reading and identifying numbers and determining their potential significance. It may be important to have a visual representation of analyte levels that may be easily accessible, visible, easy to understand, and adjustable.

While one or more embodiments are described in connection with the analyte level or concentration being a blood glucose reading, not all embodiments are limited to blood glucose readings. The term “analyte” as used herein is a broad term and is to be given its ordinary and customary meaning to a person of ordinary skill in the art (and is not to be limited to a special or customized meaning), and furthermore refers without limitation to a substance or chemical constituent in a biological fluid (for example, blood, interstitial fluid, cerebral spinal fluid, lymph fluid, or urine) that can be analyzed. Analytes can include naturally occurring substances, artificial substances, metabolites, and/or reaction products. Unless expressly disclaimed or stated otherwise, the subject matter described herein extends to other types of analyte measurements, such as cholesterol (e.g., high density lipoprotein, low density lipoprotein), lipids, bilirubin, albumin, red blood cell count, white blood cell count, creatinine, concentration of medication, concentration of vitamins, concentration of minerals, or the like.

The terms “sensor reading” or “sensor data”, as used herein is a broad term and is to be given its ordinary and customary meaning to a person of ordinary skill in the art (and are not to be limited to a special or customized meaning), and furthermore refers without limitation to any data associated with a sensor, such as a continuous analyte sensor. Sensor data includes a raw data stream, or simple data stream, of analog or digital signal directly related to a measured analyte from an analyte sensor (or other signal received from another sensor), as well as calibrated and/or filtered raw data. In one example, the sensor data comprises digital data in “counts” converted by an analog-to-digital (A/D) converter from an analog signal (e.g., voltage or amps) and includes one or more data points representative of a glucose concentration. Thus, the terms “sensor data point” and “data point” refer generally to a digital representation of sensor data at a particular time. The term broadly encompasses a plurality of time spaced data points from a sensor, such as from a substantially continuous glucose sensor, which comprises individual measurements taken at time intervals ranging from fractions of a second up to longer durations, e.g., 1, 2, or 5 minutes or longer. In another example, the sensor data may include an integrated digital value representative of one or more data points averaged over a time period. Sensor data may include calibrated data, smoothed data, filtered data, transformed data, and/or any other data associated with a sensor.

The term “algorithm” as used herein is a broad term and is to be given its ordinary and customary meaning to a person of ordinary skill in the art (and is not to be limited to a special or customized meaning), and furthermore refers without limitation to a computational process (associated with computer programming or other written instructions) involved in transforming information from one state to another.

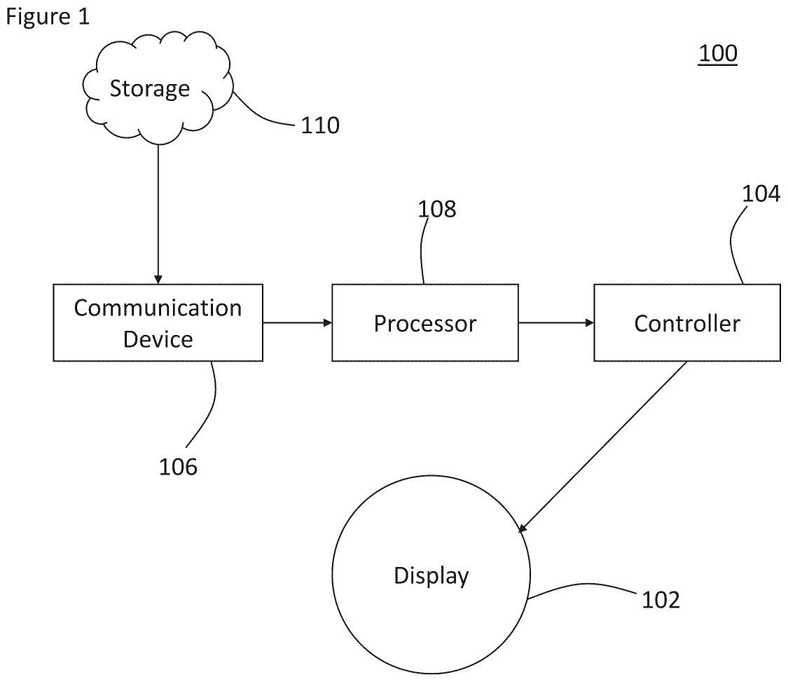

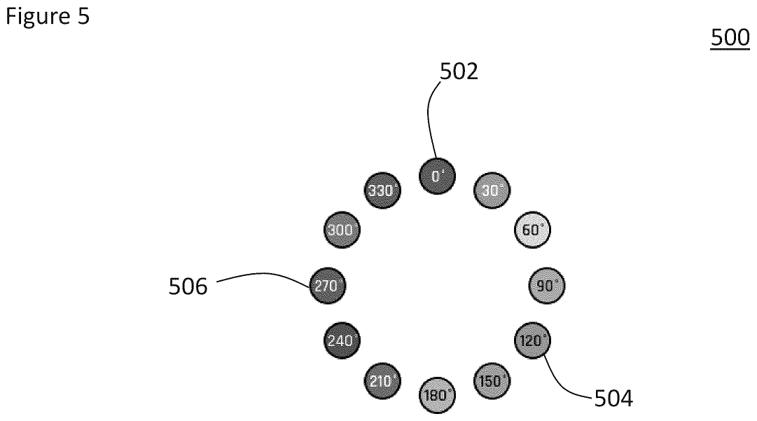

FIG. 1 illustrates one example of a system for displaying measured analyte levels. The system may include a display 102, a controller 104, a communication device 106, and a processor 108. In one example, the display may be an adjustable light display. The adjustable light display may be able to adjust a color displayed, for example a hue or a brightness displayed, discussed further below with respect to FIG. 5. The adjustable light display may be a light emitting diode (LED), a compact fluorescent lamp (CFL), a halogen lamp, a liquid crystal display (LCD), or the like. Where the display is an LCD, the display may be capable of displaying a solid color screen, as well as displaying user messages or alerts. The solid color screen and messages or alerts may be shown simultaneously (e.g., the messages or alerts may be shown on the solid color screen) or may be shown at separate times (e.g., the solid color screen may be off when the messages or alerts may be shown). The display may be adjustable in brightness, as well as adjustable in color range. The color range may be specifically adjustable to a specified hue.

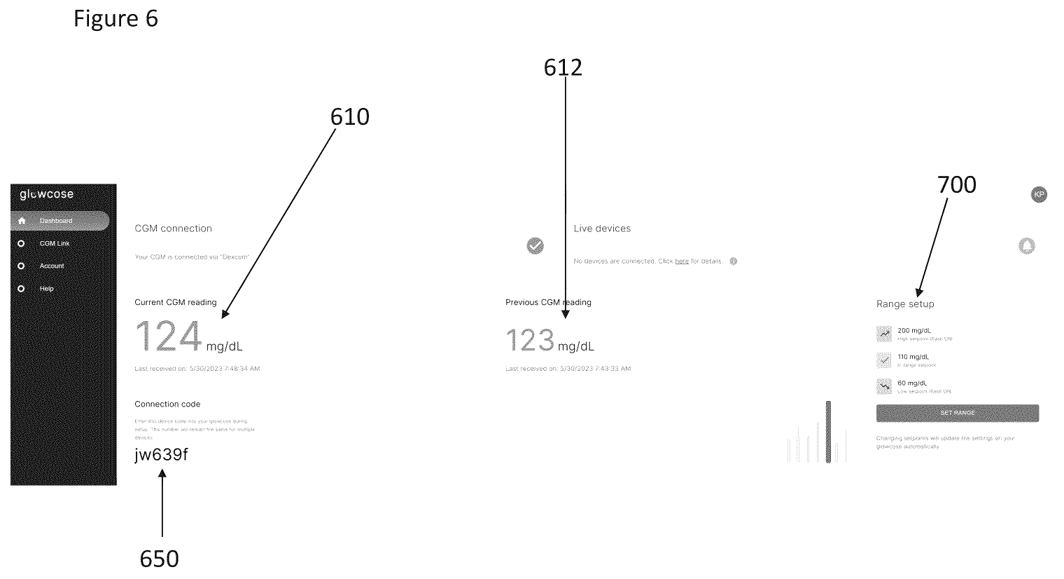

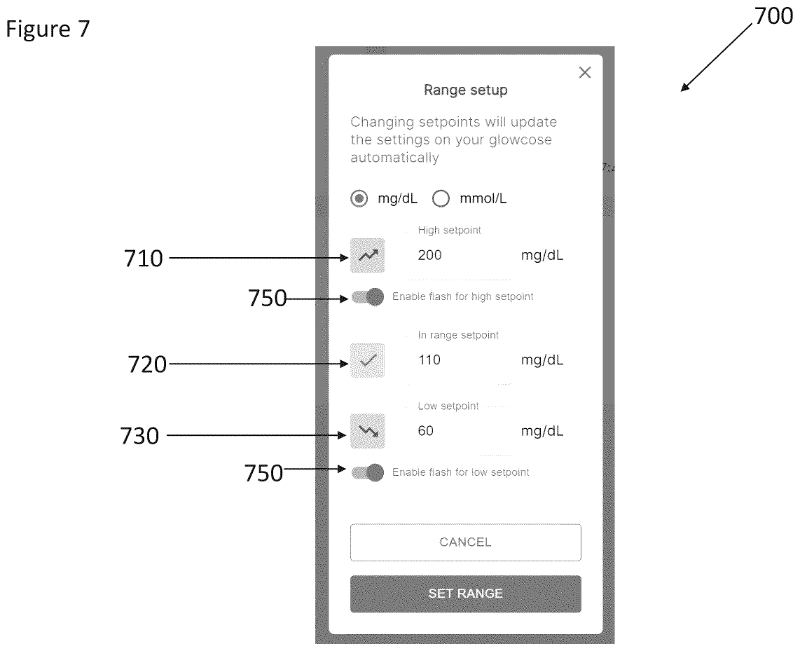

The controller of the system may locate data stored on or in a storage database 110 or stored in a web application (discussed further with respect to FIGS. 6 and 7). As used herein, the controller may include processors, microprocessors, microcontrollers, or other logic devices that operate based on instructions stored on a tangible and non-transitory computer readable storage medium, such as software applications stored on a memory. The controller may compile a database of analyte readings on a server. The database created by the controller may be sent to, or accessible by, one or more processors. As used herein, a processor may refer to a processor module, microprocessor, a computer system, a state machine, and the like that performs arithmetic and logic operations using logic circuitry that responds to and processes the basic instructions that drive a computer.

In one example, the database created by the controller may be converted from analyte readings to color values to be displayed by the display. The database of color values may then be sent to, or accessible by, one or more processors. In one example, the controller may be able to access readings from a third-party sensor, a third-party database, or multiple third-party sensors or databases. This may allow the controller to have a wider breadth of readings to have access to. For example, the user may select the desired readings for the controller to access, such as the user's CGM reading database. This may allow the controller to provide readings across different CGM platforms. The system may be compatible with multiple CGM or analyte reading systems, as well as user input data.

In the example illustrated in FIG. 1, the storage database may be a cloud storage system 110. The data may be related to a measured analyte level or concentration. In one example, the measured analyte level may be a measured blood glucose level. The storage system may communicate the measured analyte level or concentration to the controller and/or the processor via a communication device 106. The communication device may include or represent an antenna (along with associated transceiver hardware circuitry and/or software applications) for wirelessly communicating with other devices or systems. Optionally, the communication device may communicate via one or more wired connections. The communication device may communicate with one or more components of the system or with systems that are remote or independent from the system (e.g., a CGM system, another sensor, another database, or the like).

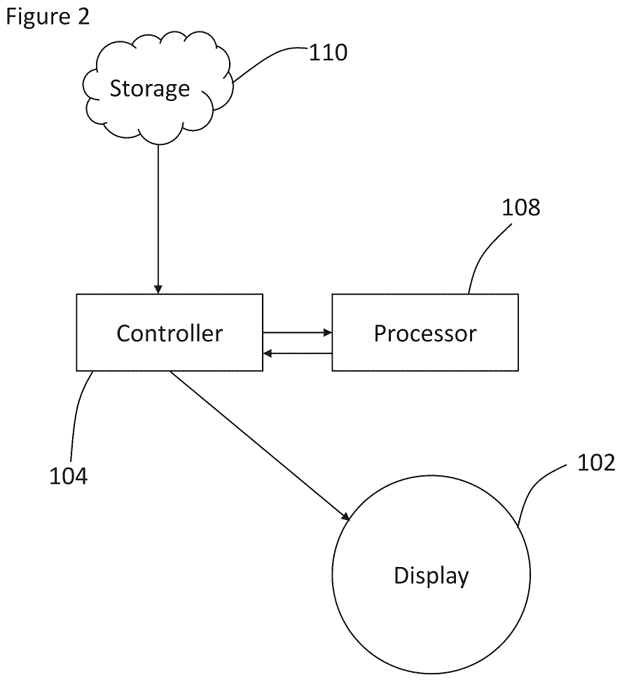

As illustrated in FIG. 2, the system may operate without the communication device. In this example, the controller may communicate directly with the storage, the processors, and the display. The controller may send the data obtained from the storage database or web application related to the measured blood glucose level to the processors. The controller may send the data to the processor via a Wi-Fi signal, a radio frequency, Bluetooth, cellular signal, or the like. In one example, rather than the controller sending the data to the processor, the processor may retrieve or fetch the data from the controller or the storage.

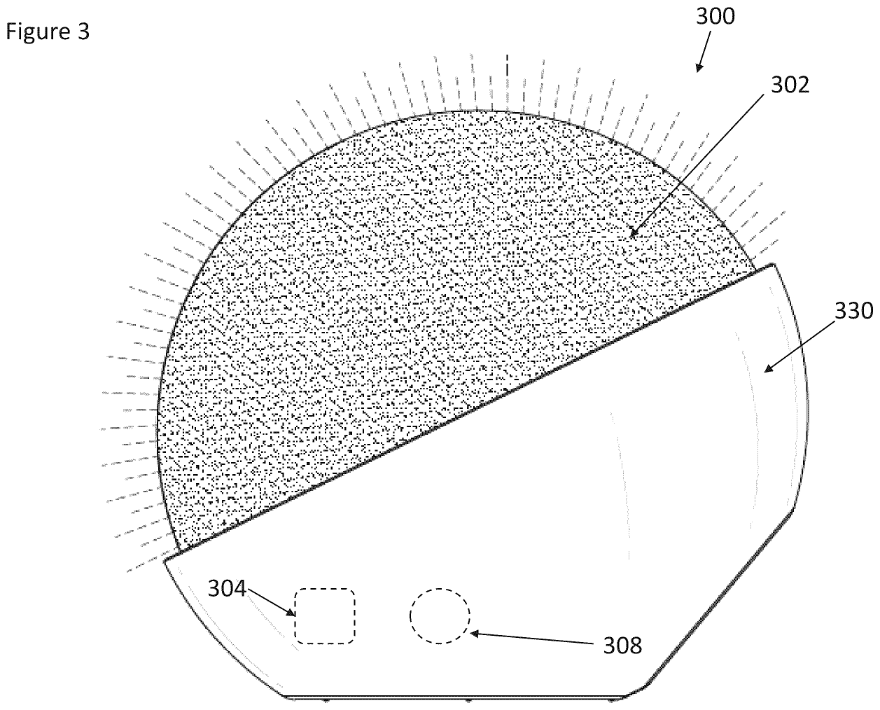

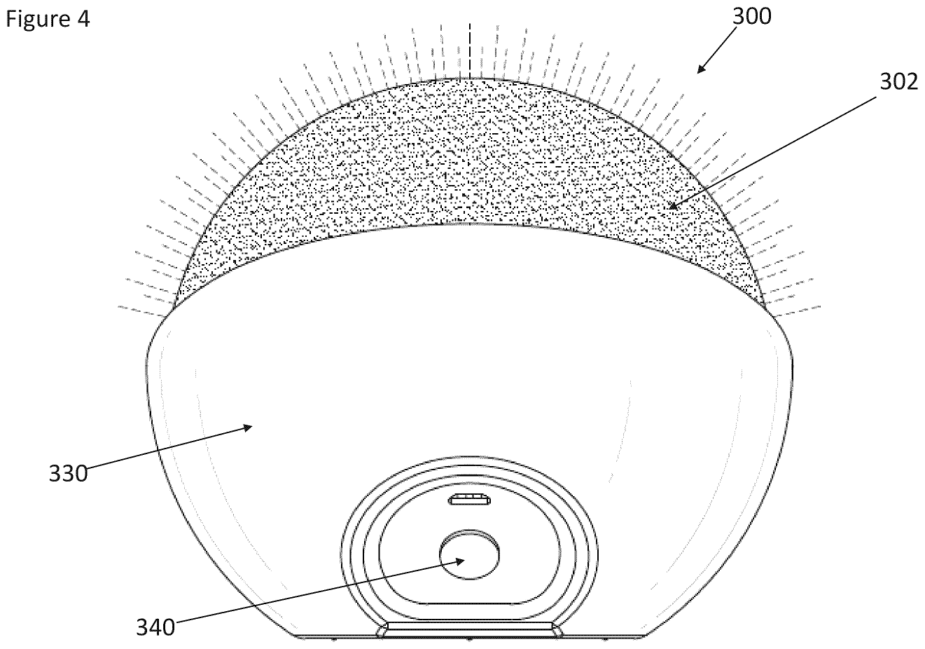

FIG. 3 illustrates a side view of a display 300 for displaying measured analyte levels, according to one example. FIG. 4 illustrates a rear view of the display 300 shown in FIG. 3, according to one example. The display illustrated in FIGS. 3 and 4 may be the display represented schematically in FIGS. 1 and 2. The display may include a light 302, a controller 304, and one or more processors 308. In one embodiment, the controller and the processor may be separate from the display. The display may include the light 302 and a base portion 330. The base portion may be a non-illuminating portion of the display. However, in another example, the base portion may be configured to illuminate with the light. In another example, the base portion may be designed such that the base portion may display numerical or textual messages. For example, the base portion may include a time, a date, a warning message, a notification, a headline, a measured analyte reading, or the like. As illustrated in FIG. 4, the base portion may include user interactable buttons 340 or touch pads. The buttons may allow the user to reset the display, silence an alarm or notification, increase/decrease a brightness, turn on/off, or the like.

The light displayed by the display may be adjustable in hue and brightness. The hue may be determined in terms of degrees, as found on a color wheel or color spectrum. Figure illustrates one example of a color wheel 500. The color wheel may follow the color spectrum of red, orange, yellow, green, blue, indigo, violet/purple, as well as the associated colors in between. In the example shown in FIG. 5, the display may illuminate a red light 502 at 0 degrees. The red light at 0 degrees may be a solid or completely red hue. At 359 degrees, just before the hue returns to 0 degrees, the light may display a substantially red hue, but may have slightly lighter hue. The light may display a green light 504 at 120 degrees. The green light at 120 degrees may be a solid or completely green hue. The light may display a purple light 506 at 270 degrees. The purple light at 270 degrees may be a solid or completely purple hue. In one example, the purple light may be displayed at 290 degrees. Additionally, the light may be adjustable at any degree on the color wheel between and 359 degrees. For example, at 30 degrees the light may be orange, at 60 degrees the light may be yellow, and so on as the degrees move around the color wheel.

FIG. 6 illustrates an application dashboard associated with the display. In one example, the application may communicate with the display, for example with the controller of the display. The application may include a connection code 650 that may be unique to the display in order to pair the communication between the application and the display. The connection code may designate a channel through which the display be connected to the application. The connection code may be a unique connection code that may be generated per user and may be used to setup the display. The code may be entered directly into the display to locate the user. There may be multiple connection codes for the same user. The user may have a unique connection code for each different display. For example, the user may have a first connection code for a bedroom display and a second connection code for a living room display. This may be beneficial as the user may want to customize the display based on the location (e.g., the user may want the brightness of the bedroom display to be reduced at night).

In one example, the application may be associated with one user of the display. In this way, the application may provide a current analyte reading 610, for example a current blood glucose level of the user. The current analyte reading may be the most recent reading measured. The application may also display a previous analyte reading 612 of the user. The previous analyte reading may be the immediately preceding analyte reading or measurement, however, the previous analyte reading may be an average of several previous analyte readings or measurements.

In one example, illustrated in FIG. 7, the application may allow a user to set customizable targets or ranges 700 for the measured analyte reading. The target or range may be set for each user. The target may be a number (e.g., 110 mg/dL), however, the target may be a range including two or more numbers (e.g., 90 mg/dL-110 mg/dL). The range settings may be adjustable by the user. In the example shown, the ranges may include a high level concentration target or range 710, a target level concentration 720, and a low level concentration 730.

The customizable targets or ranges displayed in the application may also include an option to enable flash 750 in response to a reading in the high level concentration target or range, as well as in the low level concentration target or range. When enabled, the display may periodically flash or ramp the brightness one or more times in response to a reading that is high or low. This may alert a user that their analyte level may be outside of the target level concentration. In one example, the display may flash a predetermined number of time. In another example, the display may flash until the analyte level returns to the target level concentration.

In one example, the measured analyte level may be a measured blood glucose level. A user may have multiple levels or ranges corresponding to their blood glucose level. For example, each user may have a target blood glucose level, a low blood glucose level, and a high blood glucose level. For example, a given user may have a target blood glucose level of 110 mg/dL or 6.105 mmol/L, a low blood glucose level of 70 mg/dL or 3.885 mmol/L, and a high blood glucose level of 200 mg/dL or 11.1 mmol/L. The low blood glucose level may indicate a risk of the effects of hypoglycemia. The high blood glucose level may indicate a risk of the effects of hyperglycemia. Both hypoglycemia and hyperglycemia may have harmful and potentially deadly consequences if left unattended.

The processors may receive, interpret, translate, and/or evaluate the measured analyte level received from the controller. The processor may translate the measured analyte level into a color to be displayed by the display. Specifically, the processor may determine a hue associated with the measured analyte level. In one example, the target analyte level may be displayed by the light at 120 degrees, or a completely green hue. The low analyte level may be displayed by the light at 0 degrees, or a completely red hue. The high analyte level may be displayed by the light at 290 degrees, or a completely purple hue. However, the users analyte levels may have more variability and fluctuation than three indicator points. Thus, the processor may receive the users measured analyte level and interpret what hue of light to display when the measured analyte level is not at the target, low, or high value. Each slight change in analyte level, for example blood glucose level (e.g., 1 mg/dL), may be associated with a different hue (e.g., a different degree) on the display. This allows for a customizable, individualized, and easily identifiable way to receive analyte levels.

For example, in reference to the user's ranges mentioned above, a measured blood glucose value may be 95 mg/dL at a given time. This measured value is below the target value (110 mg/dL) but above the low value (70 mg/dL). The processor may run an algorithm to determine that the hue should be set at 78 degrees for this user when their blood glucose reading is 95 mg/dL. The processor may convert the hue to RGB setting and communicate the RGB setting to the controller. The controller may then direct the light to operate with an RGB value equivalent to a hue of 78 degrees.

The processor may have an algorithm to calculate the hue associated with the measured blood glucose reading. The hue may be determined after first evaluating whether the incoming blood glucose reading is at, above, or below the user's target value. This determination may allow for a user to have a maximized range or spread above or below the target value. The maximized range may be beneficial as it may allow for a greater number of hues to be used in the range or spread. The greater number of hues used may allow for a more easily identifiable and distinguishable color pattern along the color bandwidth for the user. Said another way, the more fine-tuned the range or spread around the target, the more customizable the adjustable the color hue may be for the user.

If the blood glucose reading is equal to or greater than the target value, the value of the hue will be between 120 and 290 degrees. If the blood glucose reading is at the target value, the hue will be 120 degrees. If the blood glucose reading is equal to or less than the target value, the value of the hue will be between 0 and 120 degrees. The processor may use a different algorithm or formula once it is determined whether the blood glucose reading is above or below the target value, respectively. See the below formulas for reference. Hue (low) shows a possible formula when the blood glucose reading is at or below the target value. Hue (high) shows a possible formula when the blood glucose reading is above the target value.

$$hue ( low ) = \frac{( measured blood glucose - low value ) \times ( 120 ° - 0 ° )}{( target value - low value ) + 0 °} \\ hue ( high ) = \frac{( high value - measured blood glucose ) \times ( 290 ° - 120 ° )}{( high value - target value ) + 0 °}$$

Provided below is an example hue calculation. In the formula below the user low value=70 mg/dl, the user target value=110 mg/dl, and the user high value=200 mg/dl. In the formula below, the measured blood glucose reading is 95 mg/dL, which is below the target value (110 mg/dL), thus the hue (low) formula is used:

$$hue = \frac{( 95 - 70 ) \times ( 120 ° - 0 ° )}{( 110 - 70 ) \times 0 °} = 75 °$$

For the above discussed user, a blood glucose reading of 95 mg/dL is associated with a hue of 75 degrees.

The display may have a sensory notification component. The sensory notification component may be designed such that it may draw the attention of a person nearby the display. In one example, the sensory notification component is a flash or strobe component. For example, where the measured blood glucose is below the low value or the low level concentration range, the display may periodically flash or ramp the brightness one or more times. When the measured blood glucose may be above the high value or the high level concentration range, the display may also periodically flash or ramp the brightness one or more times. The flashing may occur periodically, for example 3 flashes every minute, or the flashing may occur constantly until the measured blood glucose level may be within the target range or may be above the low level concentration range. The flashing may draw attention to the display to indicate a measurement in either the low or high level concentration range. Additionally, the brightness may be turned to a maximum brightness during the flashing event where the low or high level concentration range is detected, received, or measured. The same flashing and brightness events may occur in response to the measured blood glucose reading being above in the high level concentration range. In other examples, the sensory notification component may include an audible alert, a tactile alert, a vibration component, a visual alert, an olfactory alert, or the like.

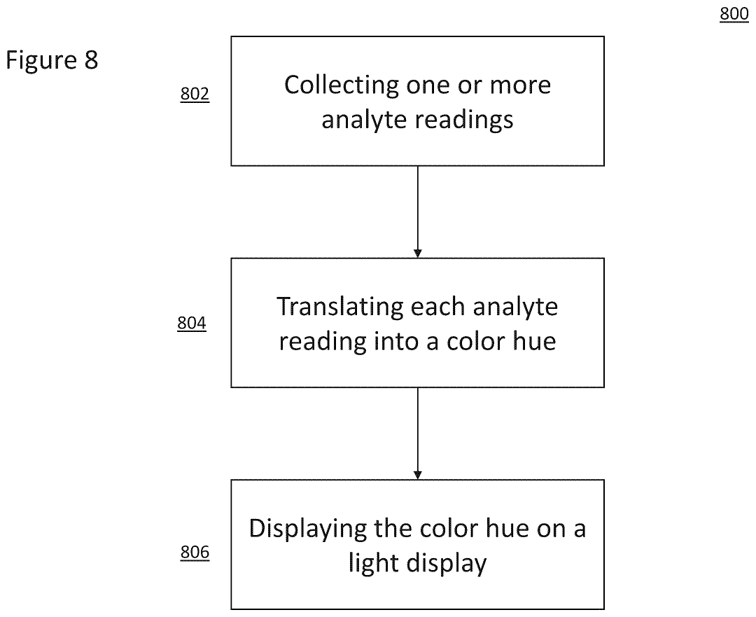

FIG. 8 shows a flowchart illustrating a method 800 for displaying analyte levels. At step 802, the method may include collecting one or more analyte readings. In one example, the analyte readings may be blood glucose readings. The blood glucose readings may be measured by a CGM. The method may collect the analyte readings by locating data stored on or in a storage database or stored in a web application. The method may compile a database of analyte readings on a server.

At step 804, the method may include translating each analyte reading into a color hue. The color hue may be determined in terms of degrees, as found on a color wheel. The analyte readings may be customized for a given user. For example, the user may have a target analyte level, a low analyte level, and a high analyte level. The color hue may indicate, on a gradient scale, where the user's analyte levels are at a given time.

At step 806, the method may include displaying the color hue on a light display. The light display may be an LED, a CFL, a halogen lamp, an LCD, or the like. Additionally, the light display may be adjustable such that the light display can illuminate a wide spectrum of color hues. The light display may display the color hue determined based on the analyte reading. This may allow for the user to be able to quickly and easily identify the user's analyte levels by viewing the light display.

Based on the color or hue of the light, the user may be able to have a quick and precise idea of their measured blood glucose levels without having to pull out their mobile device and open the CGM app. Additionally, the user's blood glucose levels may be communicated without numbers. This may be advantageous for children or diabetics with children, as a child may be able to interpret the color of the light while not being able to read and interpret large numbers. For example, if a child sees the light is red, the child may know that assistance is needed.

If the user's data is determined to be non-existent, not current, or otherwise not available the light may be switched to a white color. The white color may be set so as to not confuse the user. It may be immediately clear that there is an issue with the reading or measuring system.

The system may be entirely within the display itself. That is, the controller and processor may be in the display. However, in one example, the controller and processor may be a part of an application rather than within the display. Thus, the measure blood analyte level may not be gathered by the display. Rather, the controller would gather the measured analyte level and communicate it to the processor as part of an application. The controller may then output the color value to the display. In this example, none of the user's analyte level readings are stored on the display, only the color value. This may be advantageous to further obscure user data. Additionally, this may allow or single location adjustment of user targets. For example, the user may be able to go to the application to adjust and customize user targets without having to physically interact with the display, as discussed with reference to FIG. 7. This system may make expansion of features simpler and intuitive. For example, updates may be implemented by application software updates rather than device firmware updates. In one example, the application may be associated with the CGM. The application may directly receive the measured analyte levels from the sensors, and the application may convert the measured analyte levels into a corresponding hue. The application may then communicate the corresponding hue to the display.

In one example, a user display may be used as the display. The user display may include written or visual instructions, audible instructions, or the like.

In one embodiment, a method is provided that includes determining an analyte concentration. The method may further include determining concentration ranges based on the analyte concentration. The concentration ranges may include a low level concentration range, a target level concentration range, and a high level concentration range. The method may include communicating the one or more concentration ranges to an adjustable display. The method may include changing a hue displayed by the adjustable display based on the concentration range.

In one example, the concentration ranges may be adjustable. The method may include flashing the hue displayed by the adjustable display on and off responsive to the analyte concentration being below a predetermined threshold. The method may include sending a notification responsive to the concentration ranges being in either of the low level concentration range or the high level concentration range. In one example, the analyte concentration may include a blood glucose concentration. In one example, the hue may be measured as degrees on a color spectrum.

In one embodiment, a method is provided that includes determining an analyte concentration. The method may include communicating the analyte concentration to an adjustable display. The method may include modifying a hue displayed by the adjustable display based on the analyte concentration.

In one example, the method includes flashing the hue displayed by the adjustable display on and off responsive to the analyte concentration being below a predetermined threshold. The method may include sending a notification responsive to the analyte concentration being below a predetermined threshold.

The analyte concentration may be determined at least once every five minutes. The analyte concentration may include a blood glucose concentration. In one example, the analyte concentration may be determined by reading an output of a database. The method may include modifying a brightness of the hue displayed by the adjustable display based on a time of day.

In one embodiment, a system is provided that includes an adjustable light display and a controller. The adjustable light display may include one or more processors. The controller may be in communication with the adjustable light display. The controller may retrieve a measured analyte reading from a database and communicate the measured analyte reading to the one or more processors. The one or more processors may determine a hue of the adjustable light display based on the measured analyte reading. The controller may change the hue of the adjustable light display based on the measured analyte reading.

In one example, the controller may flash the hue of the adjustable light display on and off responsive to the measured analyte reading being below a predetermined threshold. The controller may send a notification responsive to the measured analyte reading being below a predetermined threshold. The measured analyte reading may include a blood glucose concentration.

In one example, the controller may retrieve the measured analyte reading from the database at least once every five minutes. The controller may modify a brightness of the hue of the adjustable light display based on a time of day. The controller may sync with the adjustable light display via a unique user code.

The singular forms “a”, “an”, and “the” include plural references unless the context clearly dictates otherwise. “Optional” or “optionally” means that the subsequently described event or circumstance may or may not occur, and that the description may include instances where the event occurs and instances where it does not. Approximating language, as used herein, may be applied to modify any quantitative representation that could permissibly vary without resulting in a change in the basic function to which it may be related. Accordingly, a value modified by a term or terms, such as “about,” “substantially,” and “approximately,” may be not to be limited to the precise value specified. In at least some instances, the approximating language may correspond to the precision of an instrument for measuring the value. Here and throughout the specification, range limitations may be combined and/or interchanged, such ranges may be identified and include all the sub-ranges contained therein unless context or language indicates otherwise. Moreover, unless explicitly stated to the contrary, embodiments “comprising,” “comprises,” “including,” “includes,” “having,” or “has” an element or a plurality of elements having a particular property may include additional such elements not having that property. In the appended claims, the terms “including” and “in which” are used as the plain-English equivalents of the respective terms “comprising” and “wherein.” Moreover, in the following clauses, the terms “first,” “second,” and “third,” etc. are used merely as labels, and do not impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function devoid of further structure.

Use of phrases such as “one or more of . . . and,” “one or more of . . . or,” “at least one of . . . and,” and “at least one of . . . or” are meant to encompass including only a single one of the items used in connection with the phrase, at least one of each one of the items used in connection with the phrase, or multiple ones of any or each of the items used in connection with the phrase. For example, “one or more of A, B, and C,” “one or more of A, B, or C,” “at least one of A, B, and C,” and “at least one of A, B, or C” each can mean (1) at least one A, (2) at least one B, (3) at least one C, (4) at least one A and at least one B, (5) at least one A, at least one B, and at least one C, (6) at least one B and at least one C, or (7) at least one A and at least one C.

The above description is illustrative, and not restrictive. For example, the above-described embodiments (and/or aspects thereof) may be used in combination with each other. In addition, many modifications may be made to adapt a particular situation or material to the teachings of the inventive subject matter without departing from its scope. While the dimensions and types of materials described herein define the parameters of the inventive subject matter, they are exemplary embodiments. Other embodiments will be apparent to one of ordinary skill in the art upon reviewing the above description. The scope of the inventive subject matter should, therefore, be determined with reference to the appended claims, along with the full scope of equivalents to which such clauses are entitled.

This written description uses examples to disclose the examples, including the best mode, and to enable a person of ordinary skill in the art to practice the examples, including making and using any devices or systems and performing any incorporated methods.

1. A method comprising:
determining an analyte concentration;
determining one or more concentration ranges based on the analyte concentration, the one or more concentration ranges including a low level concentration range, a target level concentration range, and a high level concentration range;
communicating the one or more concentration ranges to an adjustable display; and
changing a hue displayed by the adjustable display based on the one or more concentration ranges.

2. The method of claim 1, wherein the concentration ranges are adjustable.

3. The method of claim 1, further comprising flashing the hue displayed by the adjustable display on and off responsive to the analyte concentration being below a predetermined threshold.

4. The method of claim 1, further comprising sending a notification responsive to the one or more concentration ranges being in either of the low level concentration range or the high level concentration range.

5. The method of claim 1, wherein the analyte concentration includes a blood glucose concentration.

6. The method of claim 1, wherein the hue is measured as degrees on a color spectrum.

7. A method comprising:
determining an analyte concentration;
communicating the analyte concentration to an adjustable display; and
modifying a hue displayed by the adjustable display based on the analyte concentration.

8. The method of claim 7, further comprising flashing the hue displayed by the adjustable display on and off responsive to the analyte concentration being below a predetermined threshold.

9. The method of claim 7, further comprising sending a notification responsive to the analyte concentration being below a predetermined threshold. The method of claim 7, wherein the analyte concentration is determined at least once every five minutes.

11. The method of claim 7, wherein the analyte concentration includes a blood glucose concentration.

12. The method of claim 7, wherein the analyte concentration is determined by reading an output of a database.

13. The method of claim 7, further comprising modifying a brightness of the hue displayed by the adjustable display based on a time of day.

14. A system comprising:
an adjustable light display including one or more processors; and
a controller in communication with the adjustable light display;
wherein the controller is configured to retrieve a measured analyte reading from a database and communicate the measured analyte reading to the one or more processors,
wherein the one or more processors are configured determine a hue of the adjustable light display based on the measured analyte reading,
the controller is configured to change the hue of the adjustable light display based on the measured analyte reading.

15. The system of claim 14, wherein the controller is configured to flash the hue of the adjustable light display on and off responsive to the measured analyte reading being below a predetermined threshold.

16. The system of claim 14, wherein the controller is configured to send a notification responsive to the measured analyte reading being below a predetermined threshold.

17. The system of claim 14, wherein the measured analyte reading includes a blood glucose concentration.

18. The system of claim 14, wherein the controller is configured to retrieve the measured analyte reading from the database at least once every 5 minutes.

19. The system of claim 14, wherein the controller is configured to modify a brightness of the hue of the adjustable light display based on a time of day.

20. The system of claim 14, wherein the controller is configured to sync with the adjustable light display via a unique user code.
